(19) United States
(12) Patent Application Publication
(10) Pub. No.: US 2026/0256566 A1
(43) Pub. Date: Sep. 3, 2026

(54) INTRAURETHRAL DEVICE

(71) Applicant: INGENION MEDICAL LIMITED, London (GB)

(72) Inventors: Matthew Oliver Jones, Reading (GB); Nabeel Jan, Middlesex (GB); Edward Charles Cappabianca, Suffolk (GB)

(21) Appl. No.: 19/165,408

(22) PCT Filed: Mar. 15, 2024
(86) PCT No.: PCT/IB2024/000131

§ 371 Date: Sep. 15, 2025

Related U.S. Application Data
(60) Provisional application No. 63/452,356, filed on Mar. 15, 2023.

(51) Int. Cl. A61F 2/00

(52) U.S. Cl. CPC A61F 2/0022; A61F 2220/0008; A61F 2250/007

(57) ABSTRACT

An intraurethral device including: a valve which is configured to be actuated between an open and a closed configuration by an external magnet; a catheter coupled in fluid communication with the valve such that actuation of the valve controls the flow of fluid through the catheter; and a seal member including at least one tubular collar and at least two circumferential annular rings extending from one or more of the at least one tubular collar, wherein a first of the circumferential annular rings has a first external diameter or thickness and a second of the circumferential annular rings has a second external diameter or thickness, the first and second external diameters or thicknesses being different from each other.

### CROSS-REFERENCE TO RELATED APPLICATIONS

This application claims priority to, and the benefit of, U.S. Provisional Application No. 63/452,356, filed Mar. 15, 2023, the content of which is incorporated by reference herein in its entirety.

### TECHNICAL FIELD

Embodiments relate to intraurethral devices and, insertion systems for the placement of intraurethral devices, and the like.

### BACKGROUND

There is a desire, in general, to provide intraurethral devices, such as valves, which are capable of selectively controlling the flow of fluid therethrough. Such devices may be needed, for example, in relation to patients with urinary retention deficiencies.

An exemplary placement of such a valve in a male patient can be seen in FIG. 1, which is an extract of FIG. 3 of US6066088—reference numerals specifically referring only to prior art figures (FIGS. 1 and 2) in the present description will be presented in parentheses (reference numerals will be used (without parentheses in the description) to refer to FIGS. 3-15).

As can be seen, FIG. 1 shows a human male abdomen (30) in cross-section. A valve (1) may be located within a patient's bulbous urethra (26) generally at bulge (17) and generally upstream of the pendulous urethra (27)—in terms of the normal flow of fluid through the urethra. The valve (1) is coupled to a retention mechanism by a catheter (16) defining a lumen (21) which as an outlet (25) at the valve (1) and an inlet defined by retaining loops (28) of the retention mechanism. The retention mechanism is located in the bladder (29) and includes the retaining loops (28) which hold the retention mechanism in place relative to the bladder neck (22).

The operation of the valve (1) between open and closed configurations is intended, therefore, to control the flow of fluid from the bladder (29) through the outlet (25) and to the pendulous urethra 27.

Important advances in this regard include the intraurethral magnetic valve of WO00/02499. FIG. 1 of this document is repeated as FIG. 2 of the present application for ease of reference. The document teaches an intraurethral magnetic valve (1) for insertion in the human urethra of a person suffering from incontinence. The intraurethral magnetic valve (1) includes a nonferromagnetic cylindrical housing (2), a valve seat assembly (36) attached to one end of the housing, a spherical magnetic valve element (14) disposed for universal movement in the housing, and a ring (10) for retaining the valve element which is attached at the other end of the housing. The valve seat assembly (36) has a nonferromagnetic valve seat (4) and ferromagnetic ring (5). The valve element (14) is maintained in closed check position against the valve seat (4) by attraction to the ferromagnetic ring (5), restricting the flow of fluid through the valve (1). An external magnet is used to impart magnetic torque and attraction to the valve element (14) displacing it from the valve seat (4) and opening the valve for fluid flow. On removal of the external magnet, the valve element (14) returns to its closed check position on the valve seat (4).

The document also teaches a second embodiment in which the intraurethral magnetic valve (1) includes a mechanism having a spring for relieving excessive fluid pressure, in which the valve seat assembly (36), rather than being attached to the housing (2), is axially slidable within the housing against the force of the spring when excessive hydrostatic pressure is present to allow release of fluid through a passageway around the valve seat assembly (36).

Such advanced intraurethral magnetic valves seek to allow selective release of fluid from the bladder through use of an external magnet, in other words, a magnet external to the patient's body may be brought close to the internally fitted intraurethral magnetic valve to cause the actuation thereof from a closed configured to an open configuration.

There are, however, problems with even these intraurethral magnetic valves. For example, the valves can exhibit poor sealing around the catheter, between an outer part of the catheter and the wall of the urethra. If there is a poor seal in this area, then the valve may operate correctly to prevent the flow of fluid therethrough unless actuated to the open configuration, but fluid may leak around the catheter—particularly when the fluid pressure is relatively high.

The environment in which the valve and catheter and their retention mechanism are located can result in encrustation, for example, with a mineralised biofilm of Proteus mirabilis or other microbial material. This can result in blockage of the valve or can impact the seal between the catheter and the wall of the urethra (which may lead to leakage, as described above).

The placement of intraurethral valves has also been the subject of development. In particular, systems for the placement of intraurethral valves need to allow the valve, catheter, and an associated retaining mechanism to be pushed through the urethra into position. However, during placement in this manner, it is not uncommon for pulling and twisting of the valve, catheter, and their retaining mechanism to be required for accurate placement and navigation through the urethra. Typically, such placement systems require a portion which is manipulated by the practitioner (e.g. a surgeon or other medically qualified person) and which does not, therefore, enter the patient's urethra. All or part of this portion of the placement system needs to be removable from the valve, catheter, and their retaining mechanism after placement of the valve. Therefore, placement systems for intraurethral valve devices need to be able to navigate the valve, catheter, and their retaining mechanism into position through the urethra and need to be at least partially removable following such placement.

One such example is WO2008/067557 which teaches a system for implanting a catheter in a urethra. The system includes a catheter having a magnetic intraurethral valve disposed at one end portion of the catheter. The system also includes a tool having a tip member extending into an opening at the first end portion of the catheter to couple the tool to the catheter. The system also includes an element extending from the tool. The catheter has an internal path that provides a conduit for the element to be extended through the valve to engage a second end portion of the catheter. The tool is adapted to use the element to stress the catheter and stiffen the catheter along the length of the catheter Stiffening the catheter facilitates implantation of the catheter in the urethra by enabling pull forces, push forces, and torque forces.

Advancement in the area of intraurethral valve placement can also be found in WO2011032150. This document teaches an insertion facilitation device for facilitating insertion of a tube-like structure into a passage in a mammalian body. The device comprises: (a) an elongate stiffener including a proximal end portion and a distal end portion, the stiffener being adapted to extend in a lumen of the tube-like structure from a distal end portion thereof to a proximal end portion thereof, with the distal end portion of the stiffener at least partially within the distal end portion of the tube-like structure and the proximal end portion of the stiffener at least partially within the proximal end portion of the tube-like structure and provide a stiffness to the tube-like structure that is greater with the stiffener than without the stiffener, and be axially displaced within the tube-like structure relative to the tube-like structure; and (b) an engagement device that is adapted in an engaging condition thereof to releasably engage the stiffener and tube-like structure with the stiffener extending within the lumen of the tube-like structure such that either or both axial and radial displacement of the stiffener relative to the tube-like structure is substantially eliminated during insertion of the tube-like structure into a body passage, and in a disengaging condition thereof to release engagement of the stiffener and tube-like structure such that the stiffener is at least axially displaceable within the lumen of the tube-like structure and can be withdrawn from the tube-like structure at least so that the proximal end portion of the stiffener is in or adjacent the distal end portion of the tube-like structure.

These systems for the placement of intraurethral valves are, as can be seen from the aforementioned documents, relatively complex and there is a general desire to simply these systems.

As will be appreciated, the positioning of a valve device in a patient's urethra (male or female) can be uncomfortable. The valve device, by necessity to form a seal, presses against the wall of the urethra—see FIG. 1, for example. In addition, external forces may increase the discomfort. For example, when a patient sits, walks, runs, or climbs stairs, then there may be additional and/or different forces acting on the valve, catheter, and/or the retention mechanism. These changes in forces may alter the pressure on the urethra wall and the surrounding tissue by the valve, catheter, and/or the retention mechanism. This may increase the discomfort felt by the patient (indeed, the patient may only feel discomfort when performing some activities especially, for example, sitting). There is a need, therefore, to improve the overall comfort of such valve devices when in use. These and other problems were considered in the significant advancements of WO2020089623. However, despite these advances there remains a continual need for further advancement and improved solutions to these and other problems relating to intraurethral devices, such as valve devices.

### SUMMARY

An aspect of the present invention provides an intraurethral device including: a valve which is configured to be actuated between an open and a closed configuration by an external magnet; a catheter coupled in fluid communication with the valve such that actuation of the valve controls the flow of fluid through the catheter; and a seal member including at least one tubular collar and at least two circumferential annular rings extending from one or more of the at least one tubular collar, wherein a first of the circumferential annular rings has a first external diameter or thickness and a second of the circumferential annular rings has a second external diameter or thickness, the first and second external diameters or thicknesses being different from each other.

The at least two circumferential annular rings may include a third circumferential annular ring, wherein the first circumferential annular ring may be located remotely from the valve relative to the second circumferential annular ring and the third circumferential annular ring is located towards the valve relative to the second circumferential annular ring such that the second circumferential annular ring may be between the first and third circumferential annular rings, and wherein the third circumferential annular ring may have a third external diameter or thickness which may be different to the second external diameter or thickness.

The third external diameter or thickness may be the same as the first external diameter or thickness.

The third external diameter or thickness may be a third external diameter, the first external diameter or thickness may be a first external diameter, and the second external diameter or thickness may be a second external diameter.

The first external diameter or thickness may be a first external diameter and the second external diameter or thickness may be a second external diameter.

The third external diameter or thickness may be a third external thickness, the first external diameter or thickness may be a first external thickness, and the second external diameter or thickness may be a second external thickness.

The first external diameter or thickness may be a first external thickness and the second external diameter or thickness may be a second external thickness.

The seal member may include one or more further circumferential annular rings.

Each circumferential annular ring may be provided on the same tubular collar.

One or more of the circumferential annular rings may be provided on a different tubular collar to one or more other circumferential annular rings.

Each circumferential annular ring may include an annular groove such that each circumferential annular ring has two circumferential parts.

Another aspect of the present invention provides an intraurethral device including: a valve which is configured to be actuated between an open and a closed configuration by an external magnet; a catheter coupled in fluid communication with the valve such that actuation of the valve controls the flow of fluid through the catheter; and a seal member including a tubular collar and a first circumferential annular ring extending from the tubular collar, wherein the first circumferential annular ring includes a plurality of teeth.

The teeth may extend radially and angled with respect to a longitudinal axis of the tubular collar and/or catheter.

Each tooth may include a conical or frustoconical part remote from the tubular collar and/or the catheter.

A device may further include a second circumferential annular ring extending from the tubular collar, wherein the second circumferential annular ring includes a plurality of teeth.

A device may further include a further tubular collar and a second circumferential annular ring extending from the further tubular collar, wherein the second circumferential annular ring includes a plurality of teeth.

Another aspect of the present invention provides an intraurethral device including: a valve which is configured to be actuated between an open and a closed configuration by an external magnet; a catheter coupled in fluid communication with the valve such that actuation of the valve controls the flow of fluid through the catheter, and a seal member formed at least in part from a thermoplastic elastomer.

The thermoplastic elastomer may be styrene-ethylene-butylene-styrene.

Another aspect of the present invention provides an intraurethral device including: a valve which is configured to be actuated between an open and a closed configuration by an external magnet; a catheter coupled in fluid communication with the valve such that actuation of the valve controls the flow of fluid through the catheter; and a seal member with an outer wall at least partially defining a chamber configured to be at least partially inflated, wherein the chamber is at least partially filled with a foam material.

Another aspect of the present invention provides an intraurethral device including: a valve which is configured to be actuated between an open and a closed configuration by an external magnet; a catheter coupled in fluid communication with the valve such that actuation of the valve controls the flow of fluid through the catheter; and an extendable portion formed in a wall of the catheter by a plurality of annular undulations, such that the extendable portion can be compressed and extended by flexing of the wall of the catheter about the annular undulations.

Annular undulations may form a series of annular regions of increased external diameter with annular regions of reduced external diameter therebetween.

The extendable portion may be configured to expand radially under the force of fluid pressure within the catheter to assist in sealing the catheter against a urethra wall in use.

Another aspect of the present invention provides an intraurethral device including: a valve which is configured to be actuated between an open and a closed configuration by an external magnet; a catheter coupled in fluid communication with the valve such that actuation of the valve controls the flow of fluid through the catheter; a retention mechanism coupled to the catheter, wherein the retention mechanism is configured for deployment in a bladder; and a bladder seal or funnel which is coupled to the retention mechanism and configured to direct fluid into the catheter from the bladder and/or inhibit movement of fluid from the bladder through the urethra around the catheter.

The bladder seal or funnel may include an annular wall extending outwardly from the catheter and coupled to one or more arms or loops of the retention mechanism.

The annular wall may form a disc.

The disc may be coupled to the one or more loops or arms by one or more posts.

The annular wall may form a funnel.

Another aspect of the present invention provides an intraurethral device including: a valve which is configured to be actuated between an open and a closed configuration by an external magnet; a catheter coupled in fluid communication with the valve such that actuation of the valve controls the flow of fluid through the catheter; and one or more of: a helical protrusion extending along a length of the catheter to provide a tortuous path for fluid flowing around the catheter; a mesh protrusion extending along a length of the catheter to provide a tortuous path for fluid flowing around the catheter; and a plurality of dot protrusions extending radially and provided along a length of the catheter to provide a tortuous path for fluid flowing around the catheter, wherein the dot protrusions have a diameter and the spacing between adjacent dot protrusions is less than that diameter.

Another aspect of the present invention provides an intraurethral device including: a valve which is configured to be actuated between an open and a closed configuration by an external magnet; a catheter coupled in fluid communication with the valve such that actuation of the valve controls the flow of fluid through the catheter; a retention mechanism coupled to the catheter, wherein the retention mechanism is configured for deployment in a bladder; and a sheath configured to cover at least part of the retention mechanism during insertion of the device into a urethra.

The sheath may be dissolvable or absorbable in the urethra and/or bladder after fitting of the device.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 shows an extracted figure from WO00/02499.

FIG. 2 shows an extracted figure from US6066088.

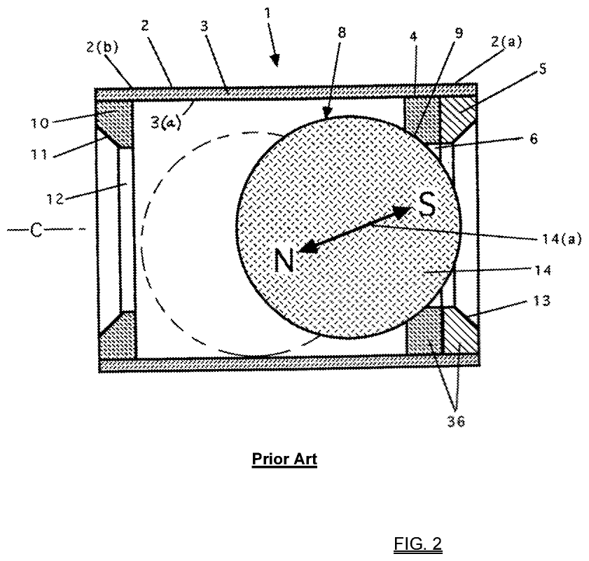

FIG. 3 shows an intraurethral device of the prior art.

FIGS. 4 and 5 show valves of the prior art.

FIGS. 6a and 6b show views of a seal member of some versions.

FIGS. 7a and 7b show views of a seal member of some versions.

FIGS. 8a and 8b show views of a seal member of some versions.

FIG. 9 shows a view of a seal member of some versions.

FIG. 10 shows a view of a seal member of some versions.

FIG. 11 shows three versions of a device with an extendable portion and/or seal member.

FIGS. 12a, 12b, and 12c show three versions of the extendable portion and/or seal member.

FIGS. 13 and 14 depict a version with a bladder seal or funnel.

FIGS. 15 and 16 depict another version with a bladder seal or funnel.

FIGS. 17 and 18 depict a version with a tortuous flow path feature in the form of a helical protrusion.

FIGS. 19 and 20 depict another version with a tortuous flow path feature in the form of a mesh protrusion.

FIGS. 21 and 22 depict another version with a tortuous flow path feature in the form of dot protrusions.

FIG. 23 shows a device with a sheath according to some versions. and

FIG. 24 shows the end of a valve according to some versions.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

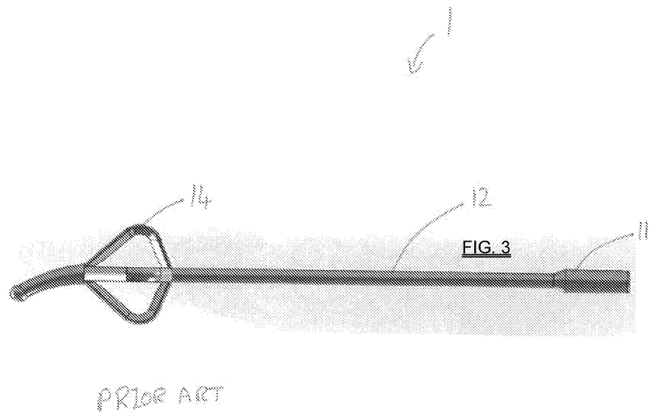

By way of overview, the present invention is generally directed to an intraurethral device comprising a unique valve. Various versions of the intraurethral device are shown and referred to in FIGS. 3 through 24 of the present application (specifically the device 1 including a valve 11). The valve 11 may be a magnetically actuated valve 11 for intraurethral placement. Therefore, the valve 11 may be an intraurethral magnetic valve 11.

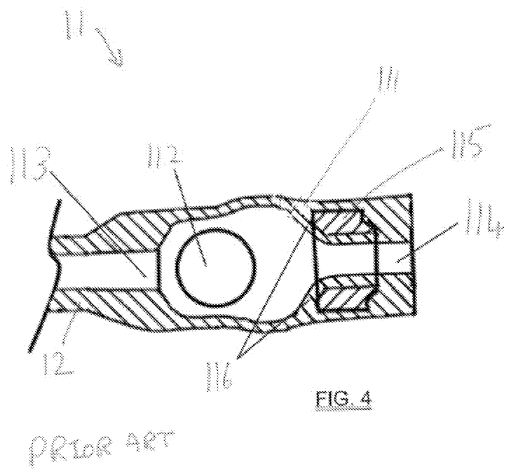

With reference to FIGS. 4 and 5 (which are depictions of prior art valves from WO2020/89623, the contents of which are incorporated by reference) by way of example, the valve 11 may include a valve main body 111 defining an internal valve volume in which is positioned a spherical magnetic valve element 112 (hereinafter “the valve element 112”). The internal valve volume has a length along a longitudinal axis of the valve main body 111. The longitudinal axis of the valve main body 111 extends from an inlet port 113 of the valve 11 to an outlet port 114 of the valve 11. The internal valve volume has a width along an axis perpendicular to the longitudinal axis of the valve main body 111.

The inlet port 113 and the outlet port 114 may both have a width (e.g. a diameter) which is less than that of the internal valve volume.

The internal valve volume is sized to receive the valve element 112, for example, as depicted in FIGS. 3 and 4. The internal valve volume may be sized to permit movement of the valve element 112 between a closed configuration and an open configuration13 as described herein.

Towards the outlet port 114, there is a ferromagnetic member 115 which may be a ring. The ferromagnetic member 115 defines a passage through which fluid may pass. The ferromagnetic member 115 is depicted in the valve 11 in FIG. 3.

The ferromagnetic member 115 may be a continuous ring of ferromagnetic material. Accordingly, the ferromagnetic member 115 may be generally tubular in form. In some versions, the ferromagnetic member 115 may be ring-like, in that a complete ring may not be formed by ferromagnetic material may form a ring-like structure formed of a plurality of ferromagnetic material segments.

The ferromagnetic member 115 may have a first end which is, when in the valve 11, towards the internal volume and a second end which is, when in the valve 11, away from the internal volume (relative to the first end). The first and second ends of the ferromagnetic member 115 may oppose each other across a length of the ferromagnetic member 115 (a length which may be parallel to the longitudinal axis of the valve 11 when the ferromagnetic member 115 is located in the valve 11).

The ferromagnetic member 115 may be coupled to the valve main body 111 and this may be achieved in several different manners.

In some versions, the valve main body 111 is formed from an elastomer material and may be formed from only the elastomer material. This elastomer material may be selected for its biocompatibility and/or the ability to impregnate the material with antimicrobial and/or antifungal agent. The ferromagnetic member 115 may be formed of a ferrous metal or ferrous metal alloy. Therefore, the valve body 111 may be formed from a relatively flexible material compared to the ferromagnetic member 115. In some versions, application of a force to the valve main body 111 may cause the valve main body 111 to deform elastically and for the internal valve volume width (in the direction of the force) to be reduced. In other words, the main body 111 may be resilient to crushing forces to which it may be exposed.

In some versions, the ferromagnetic member 115 may be embedded in a least part of the valve main body 111 with the valve main body 111 at least partially formed around the ferromagnetic member 115. In such versions, the formation of the valve main body 111 (or at least the part in which the ferromagnetic member 115 is embedded) may be a moulding process around the ferromagnetic member 115—which may, therefore, be placed inside a mould before elastomer material for the valve main body 111 (or part thereof) delivered to the mould (e.g. injected into the mould).

In some versions, the ferromagnetic member 115 may be secured by an interference fit within the valve main body 111. In such versions, the valve main body 111 may be formed separately from the ferromagnetic member 115, with a recess defined by the valve main body 111 for receipt of the ferromagnetic member 115. The ferromagnetic member 115 may then be inserted into the recess and held in place by, for example, an interference fit between the valve main body 111 and one or more surfaces of the ferromagnetic member 115. In some versions, a plug of material (which may be elastomer material such as that which may be used for the valve main body 111) may be used to secure, or help to secure the ferromagnetic member 115 in place. This plug may be located towards the outlet port 114 of the valve 11 and, indeed, may define the outlet port 114—the plug may be annular in form, for example, with the outlet port 114 defined by the plug.

In some versions, the ferromagnetic member 115 may be fitted to a spigot member of the valve main body 111. The spigot member may define a passage therethrough and the ferromagnetic member 115 may slide over the spigot member into position. A plug of material may then fit over the ferromagnetic member 115. The plug of material may be generally annular in form.

In some versions, adhesive may be used to secure, or help to secure, the ferromagnetic member 115 to the valve main body 111. In some versions, it will be understood that the spigot member may be part of the valve main body 111 and, likewise, the plug of material may form part of the valve main body 111.

In some versions, the valve main body 111 may be of a tubular form with one open end and with the ferromagnetic member 115 located at an opposing end (the opposing end forming the outlet which may be generally smaller in width than the open end). The open end may be configured to be fitted to another portion of the valve main body 111 and/or an end of a catheter 12—the other portion defining the inlet port 113 or an outlet of the catheter 12 forming the inlet port 113 for the valve 11. Therefore, the valve element 112 may be located within the valve main body 111 through the open end thereof prior to coupling to the other portion of the main body 111 and/or the end of the catheter 12 (the other portion of the main body 111 may be coupleable to the catheter 12). The catheter 12 may be integrally formed with at least part of the valve 11 (such as the main body 111), or may be adhered thereto, or may be connected using a cohesive process (such as insert molding).

In some versions in which the valve main body 111 is not moulded around the ferromagnetic member 115 during manufacture, the valve element 112 may be inserted through the outlet port 114 (or the end of the valve main body 111 at which the outlet port 114 will be located) prior to insertion of the ferromagnetic member 115 (and, for example, a plug). The elastomer material of the valve main body 111 may be configured to elastically deform to allow the insertion of the valve element 112 in this manner, for example.

The valve 11 includes a valve seat 116 which may be a non-ferromagnetic valve seat 116, for example. The valve seat 116 could be formed in a number of different manners according to versions. For example, the valve seat 116 may, in some versions, be formed by the material which forms the valve main body 111 in which the ferromagnetic member 115 is located.

The portion of the valve seat 116 which is configured to abut the valve element 112 may have a radius which matches or substantially matches an external radius of the valve element 112. In some versions, the portion of the valve seat 116 which is configured to abut the valve element 112 may be configured to deform elastically on contact with the valve element 112. Such arrangements may be configured to improve the seal between the valve seat 116 and the valve element 112.

In some versions, a separate ferromagnetic member 115 may not be used. For example, the valve seat 116 may be formed from an elastomer (e.g. silicone) including a filler material such as miniature steel or iron spheres or powder. In some versions the ferromagnetic member 115 is provided as a separate piece to the valve seat 116 but is, itself, formed from an elastomer (e.g. silicone) including a filler material such as miniature steel or iron spheres or powder.

In some versions, therefore, the end surface at the first end of the ferromagnetic member 115 may be covered by the material which forms the valve main body 111.

In some versions, the material of the valve main body 11 covers the end surface at the first end of the ferromagnetic ring 5; however, in this and some other versions, at least part of the passage through the valve 1 may be defined by the ferromagnetic ring 5 such that fluid passing therethrough may contact the ferromagnetic ring 5, for example (or at least part thereof). The valve seat 4 in these versions may be formed by the material covering the end surface at the first end of the ferromagnetic ring 5.

In some versions, with reference to FIG. 5 for example, the material of the valve main body 111 covers the end surface at the first end of the ferromagnetic member 115 but the rest of the ferromagnetic member 115 is also substantially covered by the material of the valve main body 111—such that fluid passing therethrough does not contact the ferromagnetic member 115 (or at least does not contact a substantial part thereof). The valve seat 116 in these versions may be formed by the material covering the end surface at the first end of the ferromagnetic member 115.

In some versions, a separate valve seat member is provided to form the valve seat 116. This separate valve seat member may be located adjacent the end surface at the first end of the ferromagnetic member 115 and may be secured to the ferromagnetic member 115 and/or at least partially embedded in the material of the valve main body 111. The valve seat member may be formed of a stiffer elastomer material than the valve main body 111, for example, or otherwise may have one or more properties which enhance the seal between the valve element 112 and the valve seat 116 (compared to using the material of the valve main body 111 for the valve seat 112). In some versions, the valve seat member may improve consistency in the formation of the valve seat 116 (again, compared to using the material of the valve main body 111 for the valve seat 112). The valve seat member may be formed from a different durometer silicone than the valve main body 111 (which may also be formed of silicone).

The device 1 (e.g. using the valve 11) may be actuatable between open and closed configurations (e.g. by the use of an external magnet, an external magnet being external to the device 1 and, when fitted, external to the body of the person to which the device 1 is fitted). In the open configuration, the valve element 112 is spaced apart from the valve seat 116 such that fluid may flow around the valve element 112 and through the outlet port 114. In the closed configuration, the valve element 112 is seated against the valve seat 116 such that the outlet port 114 is substantially sealed from fluid communication with the inlet port 113.

As will be appreciated, with the valve element 112 may be biased toward the closed configuration by virtue of magnetic attraction to the ferromagnetic member 115. In addition, with the device 1 (e.g. the valve 11) located in a urethra as an intraurethral device (e.g. valve) and oriented with respect to the normal flow of fluid therethrough, the inlet port 113 may be upstream of the outlet port 114 with fluid, such that the flow of fluid through the valve 11 may also assist in biasing the valve member 112 towards the closed configuration.

Actuation of the valve 11 to the open configuration may be achieved through the use of a magnetic member. The magnetic member may be positioned external to the patient to which the valve 11 is fitted and moved into relative close proximity to the valve 11 to achieve actuation from the closed to the open configuration (through expose of the valve 11 to the magnetic field of the magnetic member).

Actuation to the closed configuration may be achieved by magnetic attraction between the valve element 112 and the ferromagnetic ring 115, and may be aided by one or both of gravity and the flow of fluid through the valve 11, for example.

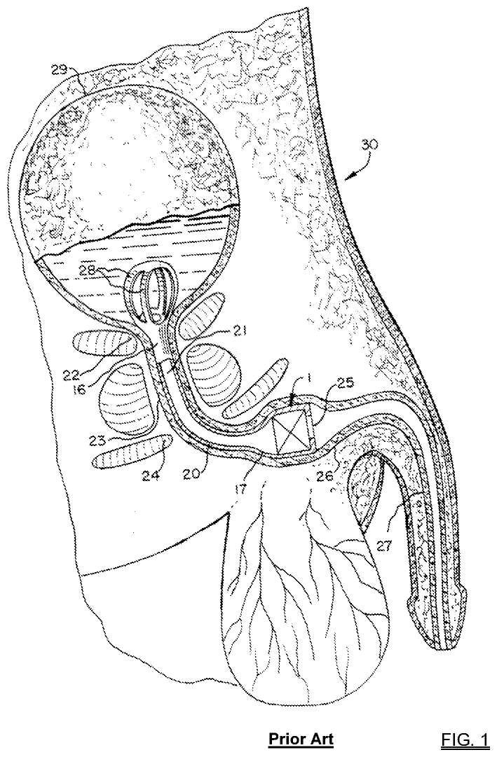

The valve 11 described herein may be fitted to a male patient or a female patient as an intraurethral magnetic valve 11. With reference to FIG. 1, the valve 11 described herein can take the place of the valve (1) shown in FIG. 1 and described in the document from which that figure was taken—see above for the document details.

As mentioned above, the valve 11 may be coupled to catheter 12. The catheter 12 provides and defines a fluid flow path to the valve 11 and, in particular, to the inlet port 113 of the valve 11. The valve 11 may be an intraurethral valve 11 and the catheter 12 may, accordingly, be coupled to a retention mechanism 14 which is configured to secure or help to secure the valve 11 and catheter 12 in the desired location. Some versions may not include a retention mechanism 14 and may be held in place by action of the catheter 12 and/or valve 11 against the urethra.

The retention mechanism 14 could take a number of different forms and is configured to be passed through a urethra to a desired location before being deployed. On deployment, the retention mechanism 14 helps to hold the catheter 12 and/or valve 11 in position. In some instances, deployment generally means the expansion or extension of at least part of the retention mechanism 14.

In some versions, the retention mechanism 14 may comprise a malecot—such as is shown in FIGS. 3, 6a, 6b, 7a, 7b, 8a, 8b, 9, 10, 11, and 15. The retention mechanism 14 may include a plurality of arms or loops or rings which act, when deployed, to restrict movement of the catheter 12 and/or valve 11 which are coupled to the retention mechanism 14. The arms or loops or rings are, therefore, moveable from an undeployed or retracted configuration to a deployed or extended configuration. Herein the arms or loops or rings are examples of deployable members of the retention mechanism 14 and this term will be used hereinafter.

In some versions, therefore, there is provided a device 1 which includes: the valve 11, the catheter 12, and the retention mechanism 14.

The length of the catheter 12 from the valve 11 to the retention mechanism 14 may be selected based on the desired placement of the valve 11—as described herein, for example.

The retention mechanism 14 may, as described above, include a plurality of deployable members. These deployable members may extend radially outwardly with respect to the catheter 12 such that they define a part of the device 1 which is wider than the catheter 12 when deployed (but which may be retracted when in the undeployed configuration to be substantially the same width as the catheter 12, for example). The deployable members may define spaces therebetween. These spaces may provide fluid communication between a volume surrounding the retention mechanism and a lumen defined by the catheter 12. This volume, in the case of an intraurethral system, may be the bladder.

Accordingly, much as in FIG. 1, the retention mechanism 14 may locate in the bladder neck with the catheter 12 extending down (in terms of normal fluid flow direction) the urethra.

The valve 11 of some versions may be inserted or otherwise implanted using the systems disclosed in, for example, WO00/02499 and/or WO2011/032150 and/or WO2020/089623, the details of which are incorporated in their entirety by reference.

The valve 11 may be a valve as described in, for example, WO00/02499 and/or WO2011/032150 and/or WO2020/089623, the details of which are incorporated in their entirety by reference.

The magnetic member may be as described in, for example, WO00/02499 and/or WO2011/032150 and/or WO2020/089623, the details of which are incorporated in their entirety by reference.

In use, therefore, the device 1 may be located in a patient's bladder and urethra. In particular, the retention mechanism 14 may be located in the bladder, with the catheter 12 extending down the urethra, and the valve 11 located along the length of the catheter 12 or at/towards an end of the catheter 12 which is remote from the retention mechanism 14 (in some versions a length of the catheter 14 also extends from the outlet port 113 of the valve 11 such that the valve 11 is located between the two opposing ends of the catheter 12.

### Sealing

In order for the valve 11 to be effective at controlling the flow of liquid (e.g. urine) through the urethra, the flow path for the liquid (or substantially all of that liquid) should be via the catheter 12 and valve 11. Accordingly, there is a need to provide a good seal between the catheter 12 and the urethra.

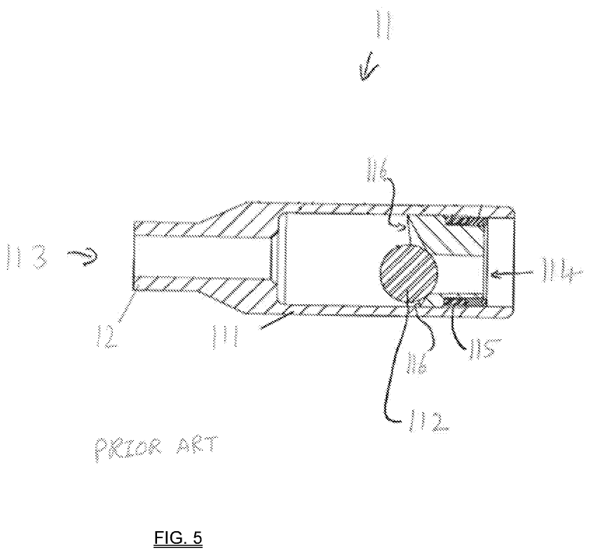

Accordingly, in some versions (with reference to FIGS. 6-12), there is provided as part of the device 1 at least one seal member 15. In some versions, there are multiple seal members 15 which may be located along a length of the catheter 12 (a length which may be between the valve 11 and the retention mechanism 14, i.e. upstream of the valve 11 in terms of the expected direction of flow of urine). If a plurality of seal members 15 is provided then there may be more than one type of seal member 15 provided—the types of which are described herein. At least one seal member 15 may be located adjacent the retention mechanism 14.

In some versions, a seal member 15 is a tubular collar 151 which fits around a length of the catheter 12. This tubular collar 151 may be slid onto the catheter 12 and secured in place through any suitable arrangement—such as welding or an adhesive, for example.

In some versions the tubular collar 151 includes a plurality of circumferential annular rings 152. These circumferential annular rings 152 may be spaced apart along a length of the tubular collar 151 and each may extend around a circumference of the catheter 12. In some versions, an external diameter of each of the plurality of circumferential annular rings 152 may be generally the same as each other circumferential annular ring 152 of the same tubular collar 151 or at least one other circumferential annular ring 152 of that tubular collar 151. Each circumferential annular ring 152 may have a cross-sectional profile (e.g. taken through a diameter of the circumferential annular ring 152 and considering one ‘end’ of the ring 152 in such a cross-section) which is rectangular or curved—see FIGS. 6a and 6b, for example. Each circumferential annular ring 152 may, therefore, form a rib.

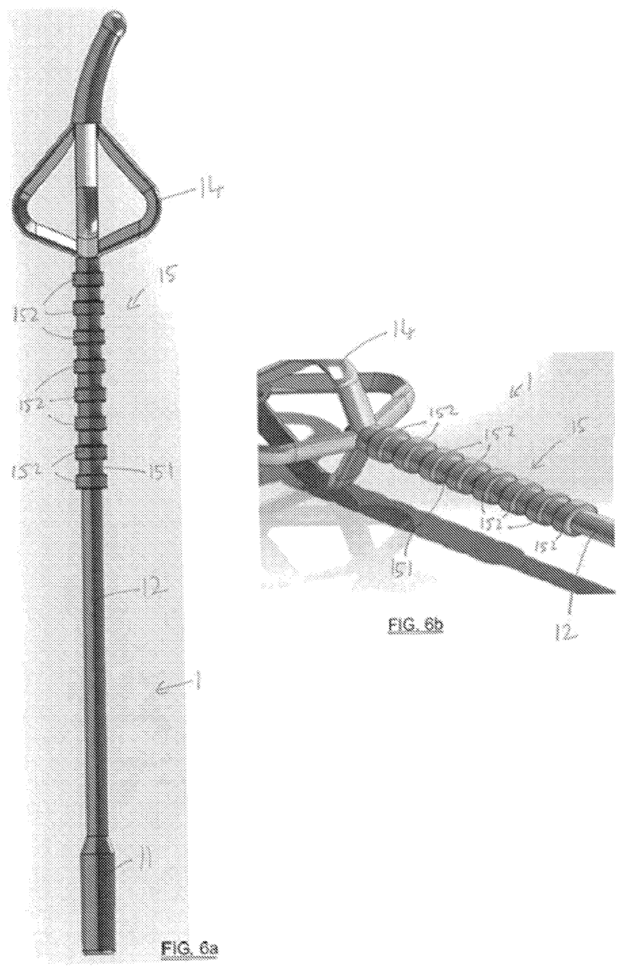

In some versions (see FIGS. 7a and 7b), the distance (e.g. height) of extension (radially) of the circumferential annular rings 152 varies along the length of the tubular collar 151—such that a circumferential annular ring 152 has a greater height of extension when compared to another of the circumferential annular rings 152. In other words, the external diameter of the circumferential annular rings 152 may be different between circumferential annular rings 152 of the tubular collar 151 (references to the relative diameters of circumferential annular rings 152 should be regarded herein equally as references to the radial height of extension of the circumferential annular rings 152). This distance of extension may be measured relative to the tubular collar 151 (or collars 151) but may be a distance of extension from a central axis of the catheter 12. In some versions, the external diameter of a first circumferential annular ring 152 towards the retention mechanism 14 is smaller than the external diameter of a second circumferential annular ring 152 which is located away from the retention mechanism 14 (relative to the first circumferential annular ring 152). Indeed, in some versions, the diameter of the circumferential annular rings 152 may increase with distance from the retention mechanism 14. In some versions, the external diameter of the circumferential annular rings 152 may reach a maximum at a first distance from the retention mechanism 14 and may then decrease for circumferential annular ring 152 which are further away from the retention mechanism 14 than the first distance. In some versions, there are an equal number of circumferential annular rings 152 either side of the circumferential annular ring 152 with the maximum diameter. In some versions, the external diameters of the circumferential annular rings 152 taper evenly with respect to each other on either side of the circumferential annular ring 152 with the maximum external diameter. In some versions, the circumferential annular ring 152 with the maximum diameter is located generally in the middle of the tubular collar 151.

In some versions, a length of extension of one circumferential annular ring 152 along the length of the tubular collar 151 and/or catheter 12 is different from another one of the circumferential annular rings 152. In other words, the thickness (along a length of the tubular collar 151 or catheter 12) may vary from one circumferential annular ring 152 to another (of the same device 1). This may, therefore, impact the degree of flexibility of the circumferential annular rings 152 and this flexibility may change (through changes in thickness or otherwise (such as by using different materials) along the length of the tubular collar 151 and/or catheter 12—with the flexibility of one circumferential annular ring 152 being different from another or each other circumferential annular ring 152. In some versions, the flexibility decreases towards the retention mechanism 14 or towards the valve 11. In some versions, the flexibility decreases towards a central circumferential annular ring 152 and then increases again (such that the flexibility is the least at the central circumferential annular ring 152). In some versions, the flexibility increases towards a central circumferential annular ring 152 and then increases again (such that the flexibility is the most at the central circumferential annular ring 152).

As will be appreciated, the circumferential annular ring 152 or rings 152 may each be described as fins in some versions.

In some versions, each circumferential annular ring 152 includes a groove (see FIGS. 7a and 7b) which may be a circumferential groove. In some versions, the groove may be located around a midline of each circumferential annular ring 152. As such, the groove may define two circumferential parts to each circumferential annular ring 152. Each of these two parts may be generally annular. This arrangement may allow the two parts to move with respect to each other during insertion and removal of the catheter 12 and may aid in retention of the catheter 12 within the urethra. In some versions, there is more than one such groove such that more than two parts may be defined.

In some versions, the thickness of the or each circumferential annular ring 152 is greatest closest to the tubular collar 151 and/or catheter 12 and the thickness decreases as that circumferential annular ring 152 extends away from the tubular collar 151 and/or catheter 12. In other words, each circumferential annular ring 152 may taper (i.e. become thinner) as that circumferential annular ring 152 extends away from the tubular collar 151 and/or catheter 12. As noted, the thickness of the circumferential annular ring 152 is the length along the tubular collar 151 and/or catheter 12 along which the circumferential annular ring 152 extends. This may assist to ease insertion of the device 1.

In some versions, each circumferential annular ring 152 has its own tubular collar 151 but the tubular collars 151 may be arranged to provide corresponding arrangements of circumferential annular rings 152 as described herein.

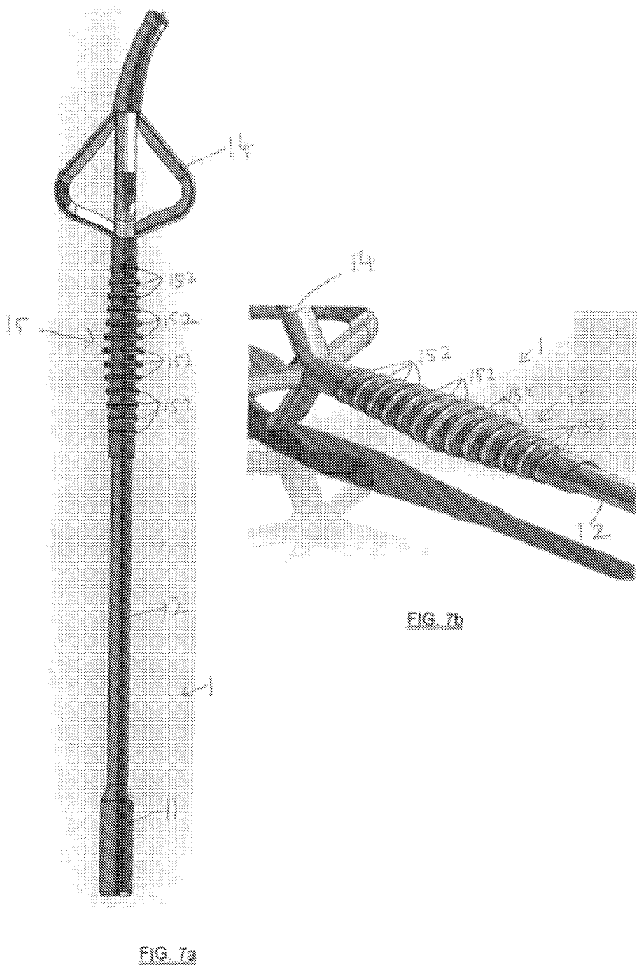

In some versions, each circumferential annular ring 152 is a generally uniform ring of constant or substantially constant radius. In some versions (see FIGS. 8a and 8b), however, each circumferential annular ring 152 may include one or more protrusions or teeth which project radially such that the radius of the circumferential annular ring 152 varies about the circumference of the tubular collar 151. Indeed, in some versions, the circumferential annular rings 152 are each a ring of protrusions or teeth. Such protrusions may have rounded ends or may have pointed ends (such that they form teeth). In some versions, the protrusions or teeth may extend radially with respect to the tubular collar 151 and/or the catheter 12. In some versions, the protrusions or teeth may be angled with respect to a longitudinal axis of the tubular collar 151 or catheter 12 such that they form barbs which resist the movement of the tubular collar 151 and/or catheter 12 in a direction away from the bladder. This may, therefore, help with retention of the catheter 12 in position. In some versions, circumferential annular rings 152 with protrusions or teeth are interspersed with one or more circumferential annular rings 152 which have a constant or substantially constant radius, to aid with sealing. In some versions, the circumferential annular rings 152 including teeth or protrusions are—as mentioned above—provided on their own tubular collars 151. The teeth may have a generally triangular shape with a point which is intended to contact the urethra. In some versions, the teeth are generally cylindrical protrusions which extend substantially radially from the tubular collar 151 and/or the catheter 12. In some version, the ends of the cylindrical protrusions which are remote from the tubular collar 151 and/or catheter 12 taper. In some such versions, each of the teeth may include a conical or frustoconical part remote from the tubular collar 151 and/or the catheter 12.

In some versions, there are two or more circumferential annular rings 152. In some versions, there are six or more circumferential annular rings 152. In some versions, there are ten or more circumferential annular rings 152. In some versions, there are fewer than twenty circumferential annular rings 152. In some versions, there are fewer than fifteen circumferential annular rings 152.

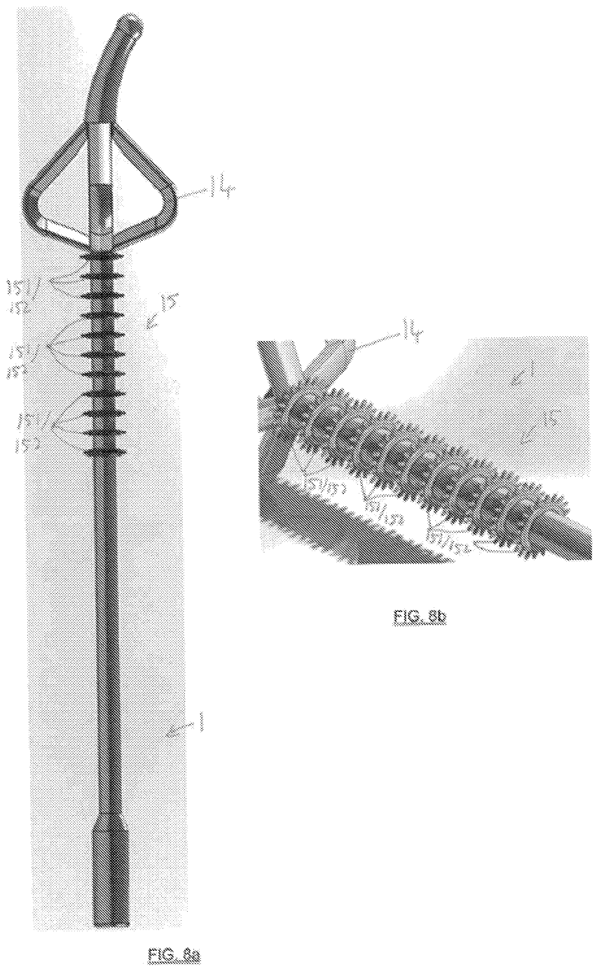

In some versions (see FIG. 9), the seal member 15 includes at least a portion which is formed from a thermoplastic elastomer such as styrene-ethylene-butylene-styrene—which may be mixed with an additive such as an oil or a filler. A seal member 15 formed of such a material may or may not include the circumferential annular rings 152. A seal member 15 formed of such a material may be a tubular collar 151 or may include a tubular collar 151 as described herein. In some versions, the seal member 15 formed of such a material may increase in diameter as the seal member 15 extends away from the retention mechanism 14 to a maximum diameter. From the region of maximum diameter, the seal member 15 may reduce in diameter towards the valve 11. The seal member 15 may be configured to conform to urethra after (and potentially during insertion) to provide a good seal between the urethra and the catheter 12.

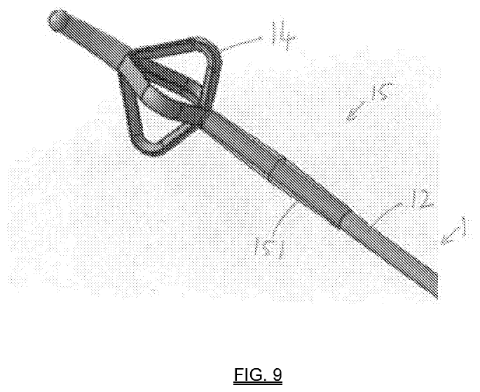

In some versions (see FIG. 10), the seal member 15 may be in the form of a tubular collar 151 which includes an outer wall 153 of a larger diameter than an external diameter of the catheter 12. The outer wall 153 may include ends which are joined (e.g. through the use of an adhesive or welding) to the catheter 12 and/or to an inner wall of the tubular collar 151 which is configured to fit around the catheter 12 (and which may be in contact along its length with the catheter 12). The tubular collar 151 of this form may, therefore, define a chamber between the outer wall 153 and the inner wall or catheter 12. That chamber may be at least partially inflated and may be a sealed chamber. In some versions, the chamber may be connected in fluid communication with an inflation tube via a chamber valve, which may be located within the chamber and which may be a one-way valve (which allows fluid into the chamber but prevents or substantially prevents the release of fluid from the chamber via the inflation tube). Accordingly, in some versions, after insertion with the chamber deflated, the chamber can be inflated, at least partially, and this may be achieved by allowing air or another fluid to pass through the inflation tube (or by pumping air or another fluid through the inflation tube). The inflation tube may be cut after insertion and inflation so that it does not extend from the urethra.

In some versions, the chamber may be filled or partially filled with a foam material. This may be an open cell foam material. The foam material may aid in the inflation of the chamber. For example, the foam material may be compressed during insertion and may be allowed to expand during inflation.

In some such versions, there may be no inflation tube.

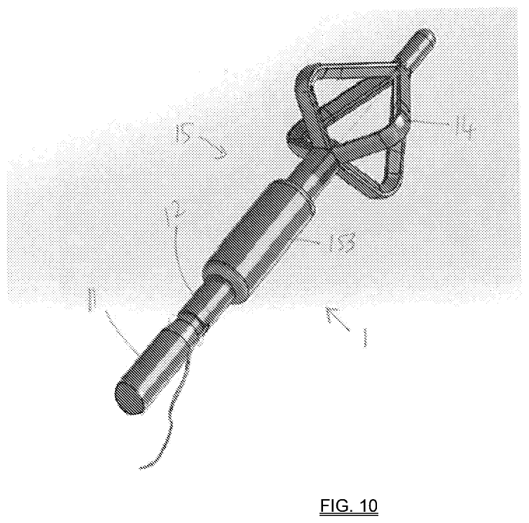

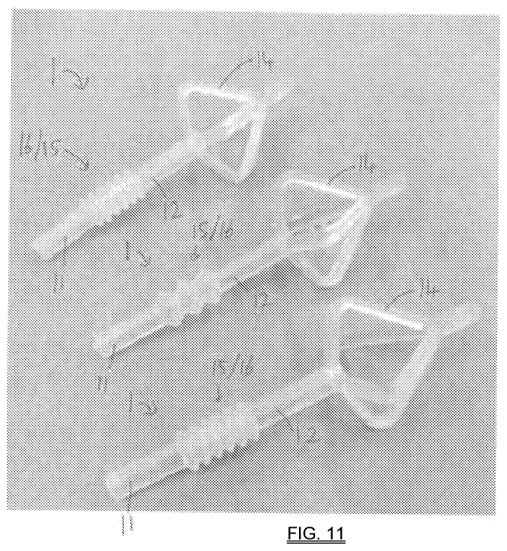

In some versions, a length of the catheter 12, which may be a length adjacent the retention mechanism 14 may include an extendable portion 16 (see FIGS. 11 and 12a-c for example). The extendable portion 16 may be formed from the wall of the catheter 12 and may comprise a series of annular undulations. The annular undulations in the wall of the catheter 12 may define annular rings about which the wall of the catheter 12 can flex, such that the extendable portion 16 can be compressed and extended (e.g. like a concertina). In some versions, the annular rings may be at least partially formed by a catheter wall which is thinner than an adjacent part. The annular undulations may, therefore, form a series of annular regions of increased external diameter with annular regions of reduced external diameter therebetween. In some versions there may be two or more such regions of increased external diameter. In some versions, there may be three or more such regions of increased external diameter. In some versions, there may be five or more such regions of increased external diameter. The length of the undulations along the catheter 12 and/or their diameter may be selected to achieve the desired flexibility and degree of extension.

The extendable portion 16 may allow for variations in the length of the catheter 12 and/or may help to ensure that the device 1 remains in position (i.e. is retained). In some versions, the extendable portion 16 may also assist in providing the seal between the catheter 12 and the urethra. Therefore, in some versions the extendable portion 16 may be considered also to be a seal member 15.

For example, the extendable portion 16 may be configured (e.g. through the thickness and/or flexibility of the material used for this portion 16 and/or the design of the annular undulations) to expand radially under the action of fluid pressure within the catheter 12. This may assist, therefore, in the sealing between the catheter 12 and the urethra—particularly when the pressure of fluid (e.g. urine) within the urethra is at its greatest).

When it comes to the removal of the device 1, the extendable portion 16 may extend under a pulling force (e.g. a manual removal force) acting on the device 1. This may extend the extendable portion 16 which may have the effect of reducing its diameter and this may therefore aid in removal of the device 1.

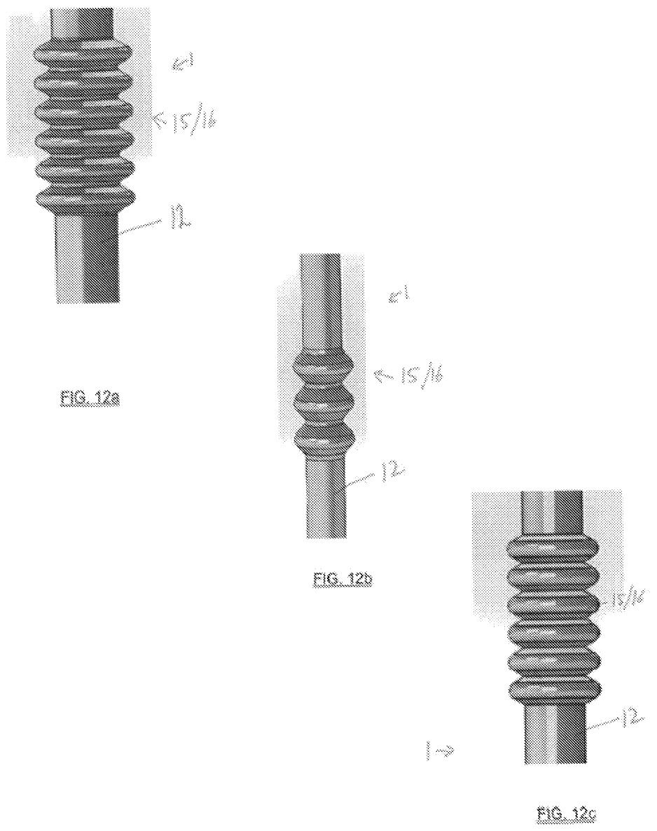

In some versions (see FIGS. 13-16), the device 1 may include a bladder seal or funnel 141. The bladder seal or funnel 141 may be configured to be located within the bladder when fitted and may serve to inhibit or prevent the flow of fluid (e.g. urine) through the urethra around the catheter 12 (rather than through the catheter 12) and/or to funnel fluid (e.g. urine) into the catheter 12.

The bladder seal or funnel 141 may be viewed as part of the retention mechanism 14 (or as a feature coupled thereto) and may be configured to be actuated therewith between a collapsed configuration and an expanded configuration. As such, the bladder seal or funnel 141 may include a wall which may be an annular wall extending outwardly from the catheter 12. The wall may be connected for movement with parts of the retention mechanism 14 (such as the loops or arms which may be part of a malecot) such that actuation of retention mechanism 14 between a retracted and extended configuration also causes actuation of the bladder seal or funnel 141 between the collapsed and expanded configuration. This may aid in insertion of the device 1 and may be achieved through the use of a guide wire which the device 1 (which may be used in this and other versions to retract and extend the retention mechanism 14).

The bladder seal or funnel 141 may be in the form of a funnel (see FIGS. 13 and 14 for example) with the wall thereof providing an annular surface which declines (in its normal fitted orientation) towards the catheter 12 to direct fluid (e.g. urine) towards catheter 12.

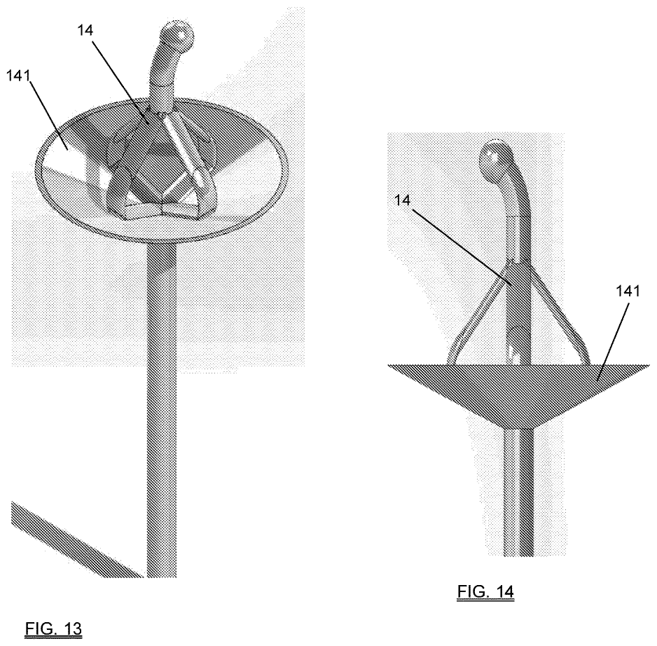

The bladder seal or funnel 141 may in the form of a disc (see FIGS. 15 and 16 for example) with the wall thereof providing an annular surface which extends radially from (i.e. generally perpendicular to) the catheter 12. This version may provide more of a sealing effect adjacent the bladder neck.

As can be seen, in some versions, the bladder seal or funnel 141 may be connected along a length of the arms or loops of the retention mechanism 14 or may be coupled thereto via respective posts. The use of the posts 142 (which extend from the loops or arms to the wall of the bladder seal or funnel 141) enable the angle of the wall of the bladder seal or funnel 141 with respect to the catheter 12 to be set—by the length of the posts 142.

Such a bladder seal or funnel 141 may be provided in combination with any other versions of the technology described herein.

### Tortuous Path

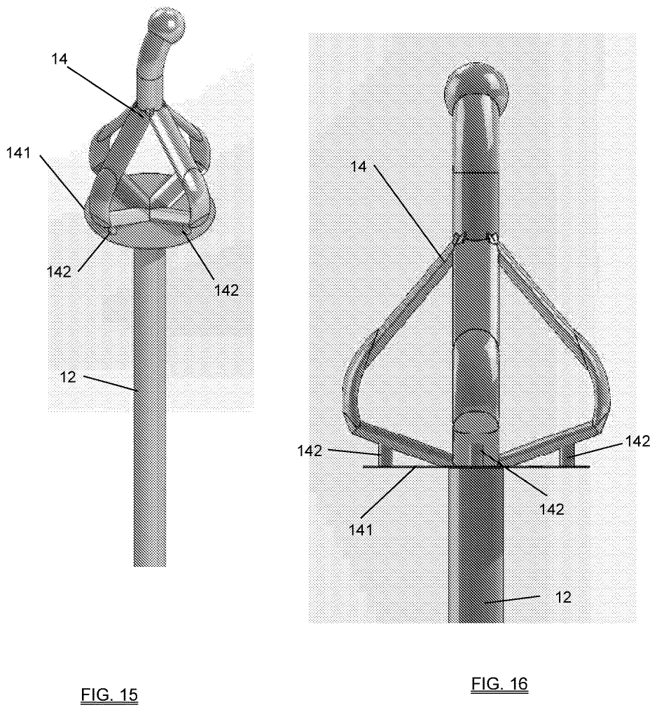

In some versions (see FIGS. 17-22, for example), the tubular collar 151 may provide a tortuous path of fluid passing through the urethra around the outside of the catheter 12 (which may be provided as the seal member 15 or in addition to any other form of seal member 15 as described herein). In some versions the catheter 12 itself may be configured to provide such a tortuous path and this may be provided in addition to another form of seal member 15 as described herein.

The configuration of the tortuous path may be described with reference to this being provided by the catheter 12 itself but the description will apply equally to the same configurations being provided as part of the tubular collar 151 or, indeed, a tubular collar 151 of several which are provided as part of the device 1.

In some versions, the tortuous path may be provided by a helical protrusion 121 (see FIGS. 17 and 18 for example) which extends around a length of the catheter 12. This helical protrusion 121 may be provided adjacent the retention mechanism 14 and/or adjacent the valve 11. In some versions, the helical protrusion 121 is provided along an entire length of the catheter 12 or substantially an entire length, and in some versions a seal member 15 may be provided over at least part of the helical protrusion 121. The helical protrusion 121 may provide a flow path for fluid (e.g. urine) from the bladder through the urethra which is longer than would otherwise be the case (e.g. longer than the path directly down the side of the catheter 12).

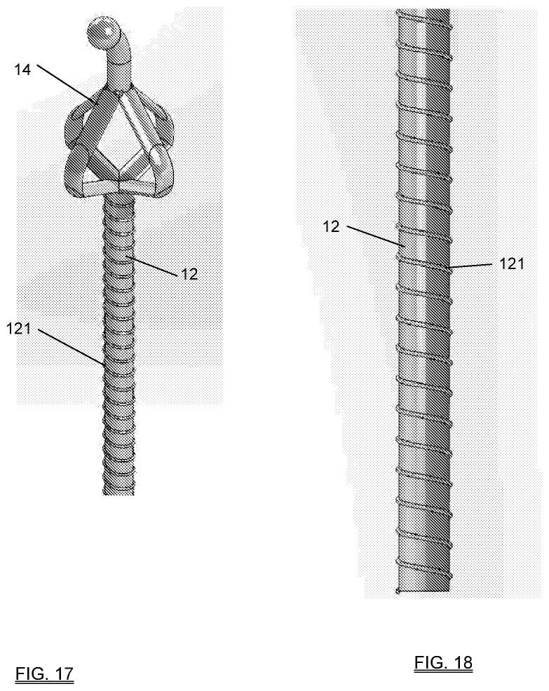

In some versions, the tortuous path may be provided by a mesh protrusion 122 which may provide a diamond shaped mesh protrusion extending from the catheter 12 (depicted in FIGS. 19 and 20, for example, as extending from a tubular collar 151 of a seal member 15).

In some versions, the mesh protrusion 122 may be formed from an inflatable member with an outer wall thereof providing the mesh protrusion or the mesh protrusion 122 may be provided on an inflatable tubular collar 151. This inflatable tubular collar 151 may be an inflatable seal member 15 as described herein (i.e. see the versions described as including the outer wall 153). In some versions, the mesh protrusion 122 may be provided as a compressible foam material or elastomer, such that the mesh protrusion 122 is deformable by contact with the urethra.

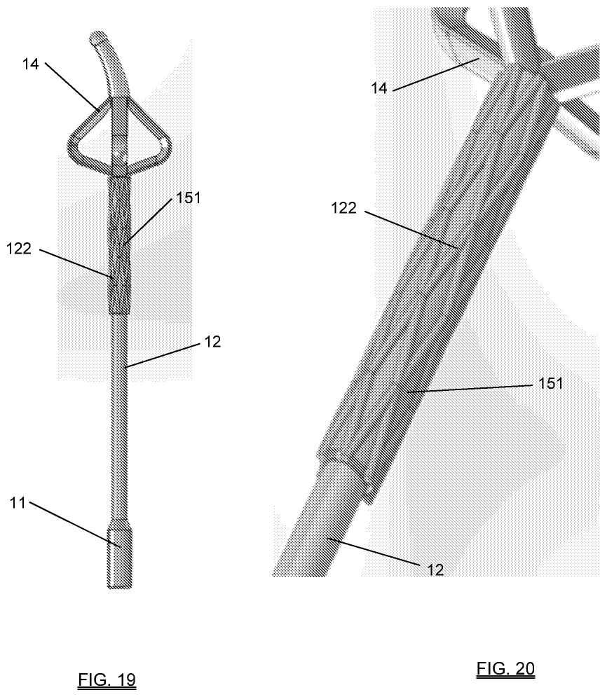

In some versions, the tortuous path may be provided by a plurality of dot protrusions 123 (see FIGS. 21 and 22, for example). These dot protrusions 123 may extend radially from the catheter 12 and/or tubular collar 151. The dot protrusions 123 may be closely spaced. For example, the spacing of adjacent dot protrusions 123 may be less than the diameter of each dot protrusions 123 (the dot protrusions 123 may all have the same diameter, for example).

This dot protrusions 123 may be provided adjacent the retention mechanism 14 and/or adjacent the valve 11. In some versions, the dot protrusions 123 are provided along an entire length of the catheter 12 or substantially an entire length, and in some versions a seal member 15 may be provided over at least some of the dot protrusions 123. The dot protrusions 123 may provide a flow path for fluid (e.g. urine) from the bladder through the urethra which is longer than would otherwise be the case (e.g. longer than the path directly down the side of the catheter 12).

The dot protrusions 123 may be formed from the same material as the catheter 12 if they are protrusions of the catheter 12, and may be formed from the same material as the tubular collar 151 if they are protrusions of the tubular collar 151.

In some versions, the tortuous path features (e.g. the dot protrusions 123, the helical protrusion 121, and/or the mesh protrusion 122 or any other suitable form of protrusion forming a tortuous path) may be formed in a moulding process (e.g. when moulding the catheter 12). In some versions, the tortuous path features may be provided by the removal (e.g. cutting or grinding) of material from the catheter 12 or tubular collar 151, or both in the form of moulding and cutting.

### Insertion and Removal

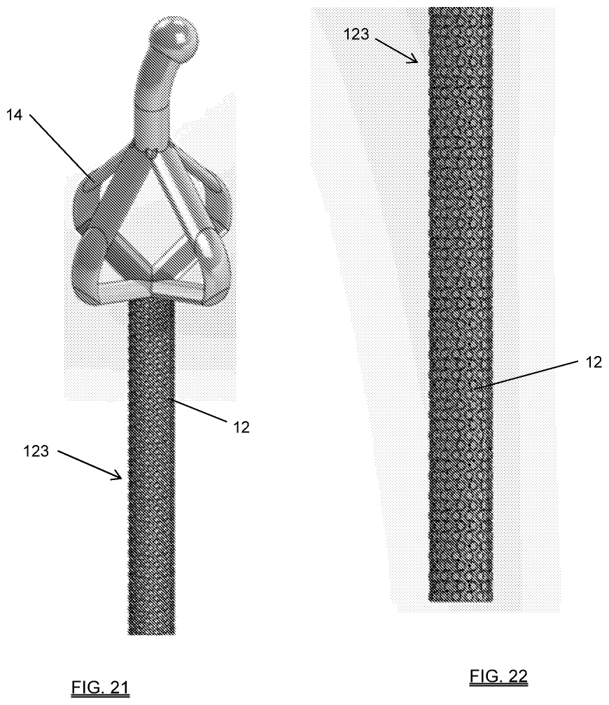

In some versions, see FIG. 23 (which is a schematic representation in the form of a cross-section without details of the valve 11 or catheter 12 being depicted, for example), the device 1 may be provided with a sheath 17. In some versions, the sheath 17 may be provided as a component of the intraurethral device 1 but in others the sheath 17 may be considered to be provided separately from the device 1—as explained herein.

In versions including a sheath 17, the sheath 17 may be provided to aid insertion. As such, the sheath 17 may present during the insertion process and may also be present shortly after insertion; however, the sheath 17 may not be present for the majority of the fitted lifespan of the device 1 (i.e. for the time the device 1 has been fitted to the patient and is operating, prior to eventual removal).

The sheath 17 may be configured to cover at least part of the retention mechanism 14 (which may be a retention mechanism 14 as described herein or some other retention mechanisms not described herein). The retention mechanism 14 may, therefore, include a bladder seal or funnel 141, for example.

The sheath 17 may be configured to aid in keeping a part or parts of the retention mechanism 14 with the retention mechanism 14 in an undeployed or retracted configuration (e.g. prior to the retention mechanism 14 changing to a deployed or extended configuration).

The sheath 17 may be in the form of a tube which fits around, in some versions, an entire circumference of the device 1, along at least part of the length of the device 1. For example, the sheath 17 may be in the form of a tube with a closed end or an open end, as described herein. In versions with a closed end, this may be located adjacent the retention mechanism 14 (and relative to the retention mechanism 14 the sealed end may be remote from the catheter 12. The closed end may be a sealed or substantially sealed end.

The sheath 17 may be formed from a flexible material or may be formed from a rigid material.

The sheath 17 may extend around all or part of the retention mechanism 14 and may extend around all or part of the catheter 12. In some versions, the sheath 17 may extend around all or part of the valve 11.

The sheath 17 may have an internal diameter which is less than an external diameter of the retention mechanism 14 when it is in its deployed configuration—such that the sheath 17 may help to restrain the retention mechanism 14 from deploying. In some versions, the retention mechanism 14 is held in its undeployed configuration by a guide wire or the like. In some versions, therefore, the sheath 17 may serve not to restrain deployment of the retention mechanism 14 but to aid (e.g. through lubrication or by virtue of a lower coefficient of friction or the like) in movement of the retention mechanism 14 through the urethra

The sheath 17 may be coated in a lubricant and/or an antiseptic and/or antibiotic material.

The sheath 17 may be formed from a dissolvable material. The material may be configured to dissolve inside the patient's body (e.g. in the urethra and/or bladder). The sheath 17 may be formed from an absorbable material. The material may be configured to absorb inside the patient's body.

In some versions the sheath 17 may be a woven structure formed from a dissolvable or absorbable material.

In some versions, the material used to form the sheath 17 may be the same as a material used for dissolvable sutures.

In some versions, the sheath 17 is formed from a polysaccharide polymer such as pullulan which may be formed into a thin-film (and which may be used at present for breath strips and drug delivery.

In some versions, exposure to urine after fitting (or during fitting) may aid in the dissolving and/or absorbing of the sheath 17.

The sheath 17 may, in some versions, be in the form of a tube with a closed end, the closed end may include one or more portions made of a dissolvable or absorbable material, with the rest of the sheath 17 formed from a material which does not readily dissolve or absorb, such that the one or more portions may weaken and break after insertion into the patient, so that the sheath 17 can be retrieved (with the device 1 retained in a fitted configuration) using, for example, one or more tethers extending therefrom to outside of the patient. These portions, therefore, allow the closed end to open (or open sufficiently to pass over the device 1).

In some versions, the sheath 17 may be configured to break or tear under force of the retention mechanism 14 changing to its deployed configuration. This may be the case in versions with or without the aforementioned dissolvable or absorbable material. The sheath 17 may have one or more lines or points or regions of weakness to allow or assist the breaking or tearing of the sheath 17 in this manner. The sheath 17 may be protected from premature breaking or tearing by a guidewire, for example, or other element which keeps the retention mechanism in its undeployed configuration.

In some versions, the sheath 17 may be fitted over the device 1 (or a part thereof) by insertion of the device 1 (or part thereof) into the sheath 17. In some versions, the sheath 17 may be cast (in a mould) around the device 1.

Accordingly, the sheath 17 may be considered to be a part of the device 1 with the device 1 prepared for insertion and potentially after insertion. However, the sheath 17 is intended to be removed for normal operation of the device 1. The sheath 17 may aid in the insertion of the device 1 into and through the urethra.

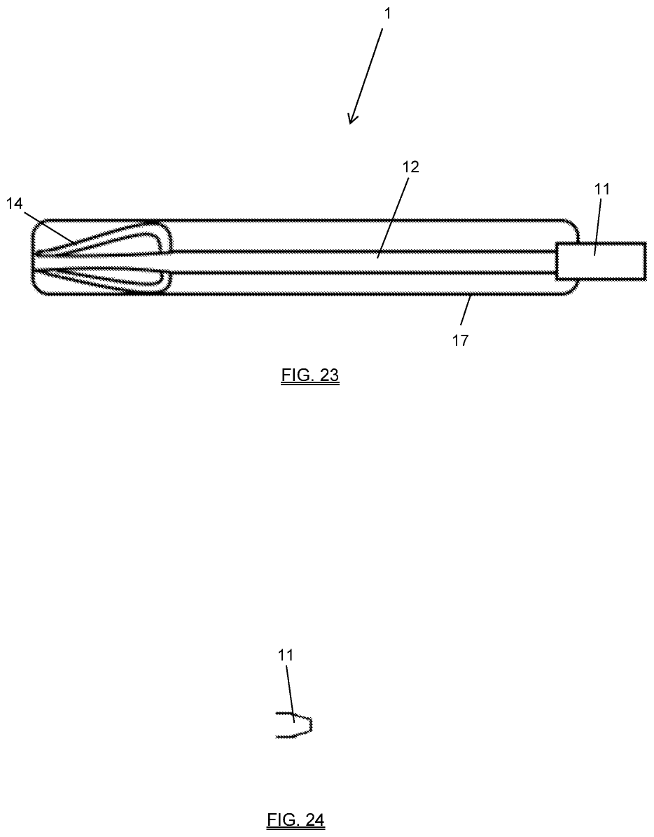

In some versions, the valve 11 may be the part of the device 1 which is remotest from the retention mechanism 14 (and so from the bladder when fitted). Accordingly, to aid in removal of the device 1, some versions may include a valve 11 with a tapered end surface. Accordingly, as the device 1 travels out of the urethra the tapered end surface may be less likely to cause discomfort or damage compared to, for example, a sharp edge between an outer circumferential surface of the valve 11 (e.g. of the valve main body 111) and an end surface of the valve 11 (e.g. of the valve main body 111). This is schematically shown in FIG. 24 (see, for comparison, the sharp edge on the end of the valve 11 of FIG. 23).

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An intraurethral device comprising:
a valve which is configured to be actuated between an open and a closed configuration by an external magnet;
a catheter coupled in fluid communication with the valve such that actuation of the valve controls the flow of fluid through the catheter; and
a seal member comprising at least one tubular collar and at least two circumferential annular rings extending from one or more of the at least one tubular collar, wherein a first of the circumferential annular rings has a first external diameter or thickness and a second of the circumferential annular rings has a second external diameter or thickness, the first and second external diameters or thicknesses being different from each other.

2. The intraurethral device of claim 1, wherein the at least two circumferential annular rings include a third circumferential annular ring, wherein the first circumferential annular ring is located remotely from the valve relative to the second circumferential annular ring and the third circumferential annular ring is located towards the valve relative to the second circumferential annular ring such that the second circumferential annular ring is between the first and third circumferential annular rings, and wherein the third circumferential annular ring has a third external diameter or thickness which is different to the second external diameter or thickness.

3. The intraurethral device of claim 2, wherein the third external diameter or thickness is the same as the first external diameter or thickness.

4. The intraurethral device of claim 3, wherein the third external diameter or thickness is a third external diameter, the first external diameter or thickness is a first external diameter, and the second external diameter or thickness is a second external diameter.

5. The intraurethral device of claim 3, wherein the third external diameter or thickness is a third external thickness, the first external diameter or thickness is a first external thickness, and the second external diameter or thickness is a second external thickness.

6. The intraurethral device of claim 1, wherein the first external diameter or thickness is a first external diameter and the second external diameter or thickness is a second external diameter.

7. The intraurethral device of claim 1, wherein the first external diameter or thickness is a first external thickness and the second external diameter or thickness is a second external thickness.

8. The intraurethral device of claim 1, wherein the seal member comprises one or more further circumferential annular rings.

9. The intraurethral device of claim 1, wherein each circumferential annular ring is provided on the same tubular collar.

10. The intraurethral device of claim 1, wherein one or more of the circumferential annular rings are provided on a different tubular collar to one or more other circumferential annular rings.

11. The intraurethral device of claim 1, wherein each circumferential annular ring comprises an annular groove such that each circumferential annular ring has two circumferential parts.

12. An intraurethral device comprising:
a valve which is configured to be actuated between an open and a closed configuration by an external magnet;
a catheter coupled in fluid communication with the valve such that actuation of the valve controls the flow of fluid through the catheter; and
a seal member comprising a tubular collar and a first circumferential annular ring extending from the tubular collar, wherein the first circumferential annular ring comprises a plurality of teeth.

13. The intraurethral device of claim 12, wherein the teeth extend radially and angled with respect to a longitudinal axis of the tubular collar and/or catheter.

14. The intraurethral device of claim 13, wherein each tooth comprises a conical or frustoconical part remote from the tubular collar and/or the catheter.

15. The intraurethral device of claim 12, further comprising a second circumferential annular ring extending from the tubular collar, wherein the second circumferential annular ring comprises a plurality of teeth.

16. The intraurethral device of claim 12, further comprising a further tubular collar and a second circumferential annular ring extending from the further tubular collar, wherein the second circumferential annular ring comprises a plurality of teeth.

17. An intraurethral device comprising:
a valve which is configured to be actuated between an open and a closed configuration by an external magnet;
a catheter coupled in fluid communication with the valve such that actuation of the valve controls the flow of fluid through the catheter; and
a seal member formed at least in part from a thermoplastic elastomer.

18. The intraurethral device of claim 17, wherein the thermoplastic elastomer is styrene-ethylene-butylene-styrene.

19. An intraurethral device comprising:
a valve which is configured to be actuated between an open and a closed configuration by an external magnet;
a catheter coupled in fluid communication with the valve such that actuation of the valve controls the flow of fluid through the catheter; and
a seal member with an outer wall at least partially defining a chamber configured to be at least partially inflated, wherein the chamber is at least partially filled with a foam material.

20.-29. (canceled)
